(19) United States
(12) Patent Application Publication
(10) Pub. No.: US 2025/0188015 A1
(43) Pub. Date: Jun. 12, 2025

(54) INDANE BIS-O-AMINOPHENOLS AND POLYMERS PREPARED THEREFROM

(71) Applicant: Fujifilm Electronic Materials U.S.A., Inc., N. Kingstown, RI (US)

(72) Inventor: Binod B. De, Attleboro, MA (US)

(21) Appl. No.: 18/954,897

(22) Filed: Nov. 21, 2024

Related U.S. Application Data
(60) Provisional application No. 63/608,468, filed on Dec. 11, 2023.

(51) Int. Cl. C07C 215/86; C08G 69/32

(52) U.S. Cl. CPC C07C 215/86; C08G 69/32

(57) ABSTRACT

There are provided indane bis-o-aminophenol compounds capable of producing soluble polyimides, functional polyimides, and poly-o-hydroxyamides.

### CROSS-REFERENCE TO RELATED APPLICATION

The present application claims priority to U.S. Provisional Application Ser. No. 63/608,468, filed on Dec. 11, 2023, the contents of which are hereby incorporated by reference in their entirety.

### TECHNICAL FIELD

This application relates to chemical compounds suitable for the preparation of polyimides and polybenzoxazoles commonly used in the in microelectronics industry.

### BACKGROUND

Dielectric material requirements for semiconductor packaging applications are continuously evolving. The trend in electronic packaging continues to move towards faster processing speeds, increased complexity and higher packing density while maintaining a high level of reliability. As electronic packaging technology advances and chips continue to shrink in size, the demand for innovative and high-performance resin compositions is growing. There is a demand for development of new monomers which are suitable for the preparation of polyimides and polybenzoxazoles that meet the challenging requirements of the microelectronics industry.

### SUMMARY

The present disclosure provides indane bis-o-aminophenol compounds having formula Ia;

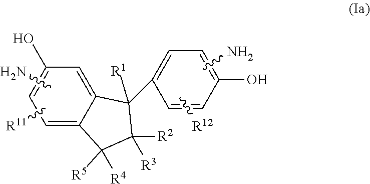

wherein each of R¹, R², R³, R⁴, and R⁵ independently, is a hydrogen atom, a substituted or unsubstituted C₁-C₁₂ alkyl, a partially halogen substituted or fully halogen substituted C₁-C₁₂ alkyl, a substituted or unsubstituted C₄-C₁₈ cycloalkyl, a substituted or unsubstituted C₆-C₂₂ aryl, or a substituted or unsubstituted C₅-C₂₂ heteroaryl; each of R¹¹ and R¹² independently is a hydrogen atom, a linear or branched C₁-C₄ alkyl, a partially halogen substituted or fully halogen substituted C₁-C₄ alkyl, a C₅-C₁₂ cycloalkyl, a C₆-C₁₈ aryl, a C₅-C₁₈ heteroaryl group, a C₁-C₄ alkoxy group, or a halogen atom.

In some embodiments, the disclosure provides compositions comprising dielectric film-forming compositions including (a) at least one polymer comprising at least one indane bis-o-aminophenol compound of formula (1a) as a diamine comonomer; and b) a comonomer derived from a dianhydride, a comonomer derived from a diacid chloride, or a combination thereof, wherein the polymer provides a dielectric layer, and when the dielectric layer is present in a microelectronic device, the modified dielectric layer imparts good reliability to the device.

### DETAILED DESCRIPTION

The disclosure provides indane bis-o-aminophenol compounds which are suitable as comonomers for the preparation of polymers that meet the challenging requirements of the microelectronics industry. In particular, the comonomers enable the preparation of fully cyclized polyimides and fully cyclized polyimides containing functional groups selected from substituted or unsubstituted linear alkenyl groups, substituted or unsubstituted linear alkynyl groups, (meth)acrylic groups, or hydroxyl groups, wherein the polyimides are soluble in commonly used formulation solvents.

In other embodiments, the comonomers of the disclosure enable the preparation of polybenzoxazole precursors which, after cyclization on a substrate, produce polybenzoxazoles with high temperature stability, wherein these polybenzoxazole precursors are soluble in commonly used formulation solvents.

Some embodiments of the disclosure provide indane bis-o-aminophenol compounds of structure Ia:

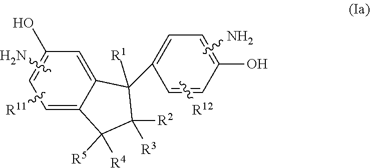

wherein each of R¹, R², R³, R⁴, and R⁵ independently, is a hydrogen atom, a substituted or unsubstituted C₁-C₁₂ alkyl, a partially halogen substituted or fully halogen substituted C₁-C₁₂ alkyl, a substituted or unsubstituted C₄-C₁₈ cycloalkyl, a substituted or unsubstituted C₆-C₂₂ aryl, or a substituted or unsubstituted C₅-C₂₂ heteroaryl; each of R¹¹ and R¹² independently, is a hydrogen atom, a linear or branched C₁-C₄ alkyl, a partially halogen substituted or fully halogen substituted C₁-C₄alkyl, a C₅-C₁₂ cycloalkyl, a C₆-C₁₈ aryl, a C₅-C₁₈ heteroaryl group, a C₁-C₄ alkoxy group or a halogen atom.

In some embodiments there are provided indane bis-o-aminophenol compounds having formula Ib:

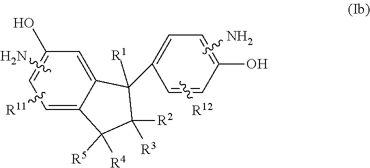

wherein each of R¹, R², R³, R⁴, and R⁵ independently, is a hydrogen atom, a substituted or unsubstituted C₁-C₁₂ alkyl, or a substituted or unsubstituted C₄-C₁₈ cycloalkyl; each of R¹¹ and R¹² independently, is a hydrogen atom, a linear or branched C₁-C₄ alkyl, a C₅-C₁₂ cycloalkyl, or a C₁-C₄ alkoxy group.

In some embodiments, there are provided indane bis-o-aminophenol compounds having formula:

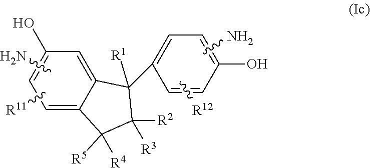

wherein R¹ or R⁴ each independently is a substituted or unsubstituted C₁-C₁₂ alkyl group; R², R³, and R⁵, are independently a hydrogen atom, a substituted or unsubstituted C₁-C₁₂ alkyl, or a substituted or unsubstituted C₄-C₁₈ cycloalkyl; and each of R¹¹ and R¹² is independently a hydrogen atom, a linear or branched C₁-C₄ alkyl, a C₅-C₁₂ cycloalkyl, or a C₁-C₄ alkoxy group.

In some embodiments there are provided indane bis-o-aminophenol compounds having formula Id:

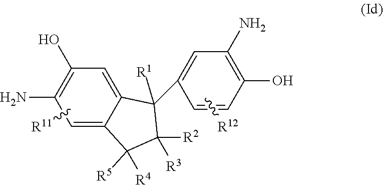

wherein R¹ or R⁴ is independently a substituted or unsubstituted C₁-C₁₂ alkyl group; R² and R³ are a hydrogen atom; R⁵ is a hydrogen atom, a substituted or unsubstituted C₁-C₁₂ alkyl, or a substituted or unsubstituted C₄-C₁₈ cycloalkyl; each of R¹¹ and R¹² independently, is a hydrogen atom, a linear or branched C₁-C₄ alkyl, a C₅-C₁₂ cycloalkyl, or a C₁-C₄ alkoxy group.

Examples of substituted or unsubstituted C₁-C₁₂ alkyl groups in R¹, R², R³, R⁴, R⁵, R¹¹ and R¹² include, but are not limited to, methyl, ethyl, propyl, isopropyl, butyl, isobutyl, t-butyl, amyl, hexyl, and 2-methylhexyl. Examples of substituted or unsubstituted C₄-C₁₈ cycloalkyl in R¹, R², R³, R⁴, R⁵, R¹¹ and R¹² include, but are not limited to, cyclopentyl, cyclohexyl, and cycloheptyl. Examples of a partially halogen substituted or fully halogen substituted C₁-C₁₂ alkyl include trifluoromethyl, pentafluoroethyl and the like. Examples of a substituted or unsubstituted C₆-C₂₂ aryl in in R¹, R², R³, R⁴, R⁵, R¹¹ and R¹² include phenyl, 4-methyl-phenyl, 2,4,6-trimethyl-naphthyl and the like. Examples of a substituted or unsubstituted C₄-C₂₂ heteroaryl; R¹, R², R³, R⁴, R⁵, R¹¹ and R¹² include furanyl; pyrazinyl, pyrrolyl and the like. Examples of C₁-C₄ alkoxy groups in R¹¹ and R¹² include methoxy, ethoxy, isopropyloxy, butoxy, and the like.

Examples of indane bis-o-aminophenol compound (Ia) include, but are not limited to:

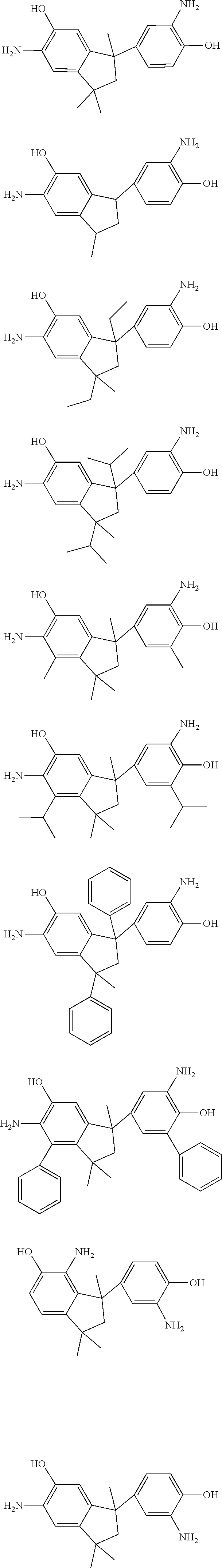

The synthesis of bisphenol compounds comprising an indane moiety can be accomplished by a facile route through a direct condensation of bisphenol compounds with sulfuric acid and subsequent conversion of the dimers to indane bisphenol using organic sulfonic acid or organic acid such as methane sulfonic acid, trifluoromethane sulfonic acid, formic acid, acetic acid and the like (Process 1). Examples of such processes through a stable 4-[2-Isopropylidene]-phenol carbocation from bisphenol A are disclosed in Wei-Fu Chen; Organic Letters, 2004 Vol. 6, No. 14 pp 2341-2343. Indane bisphenol compounds for use in the practice of this disclosure can be obtained from commercial sources and are prepared using conventional processes. Examples of such processes are disclosed in, e.g., U.S. Pat. No. 4,988,785, the entire contents of which are hereby incorporated by reference.

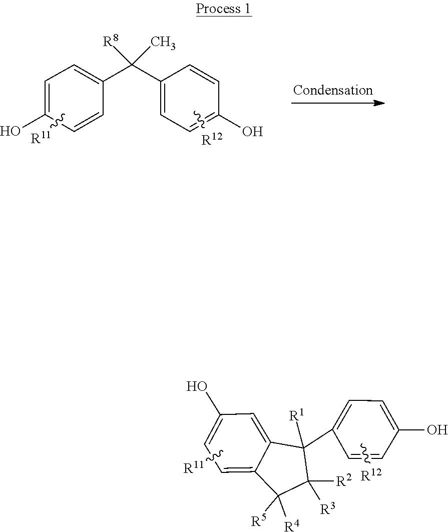

In Process 1, the condensation of bisphenol A enables production of substituted indane bisphenol compounds in a single or in a two-step process wherein R¹, R², R³, R⁴, R⁵, R¹¹ and R¹² have the same meaning described earlier and R⁸ is a hydrogen atom, a substituted or unsubstituted C₁-C₁₂ alkyl, a partially halogen substituted or fully halogen substituted C₁-C₁₂ alkyl, a substituted or unsubstituted C₄-C₁₈ cycloalkyl, a substituted or unsubstituted C₆-C₂₂ aryl, or a substituted or unsubstituted C₆-C₂₂ heteroaryl group.

Alternatively, the bisphenol compounds comprising an indane moiety can be accomplished by the conversion of the amino groups of diamino phenyl indane to hydroxyl groups by diazonium reaction forming indane bisphenol (Process 2) wherein R¹, R², R³, R⁴, R⁵, R¹¹ and R¹² have the same meaning described earlier. Examples of such processes through a stable 4-[2-Isopropylidene]-phenol from α-methylstyrene are disclosed in Masanori Terasaki Chemistry Letters 2005, Vol. 34, No. 2, 188-189.

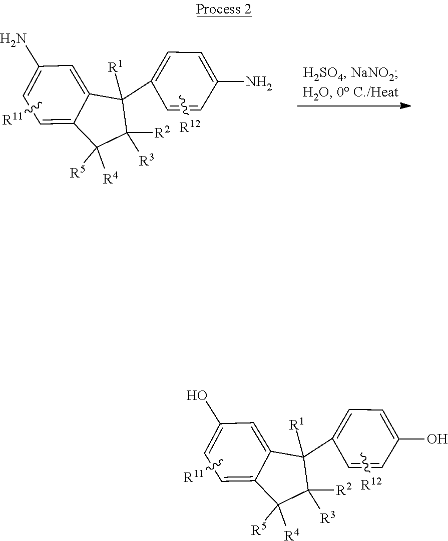

Nitration of indane bisphenols using dilute nitric acid (6-40 wt %) and subsequent reduction of nitro compounds form indane bis-o-aminophenol compounds (Process 3). Examples of such process of nitration and reduction of nitro bisphenol compounds are disclosed in, e.g., U.S. Pat. No. 6,861,560 and the entire contents of which are hereby incorporated by reference.

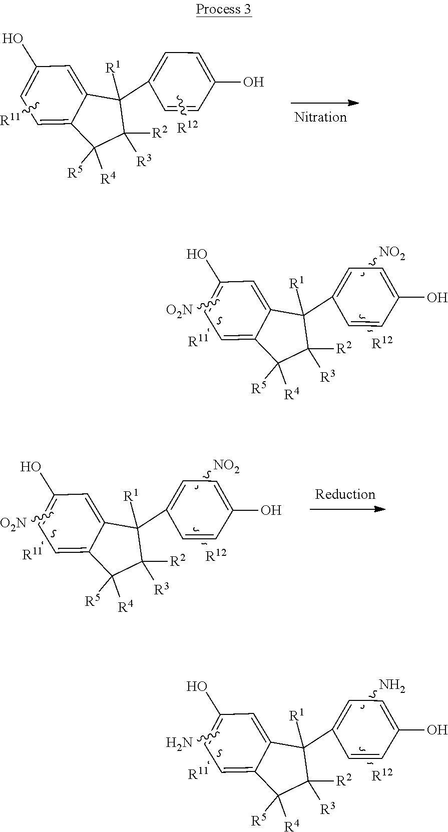

Alternatively, hydroxyl groups of indane bis-o-aminophenol compounds can be protected before nitration and hydroxyl group deprotection occurs after nitration (Process 4).

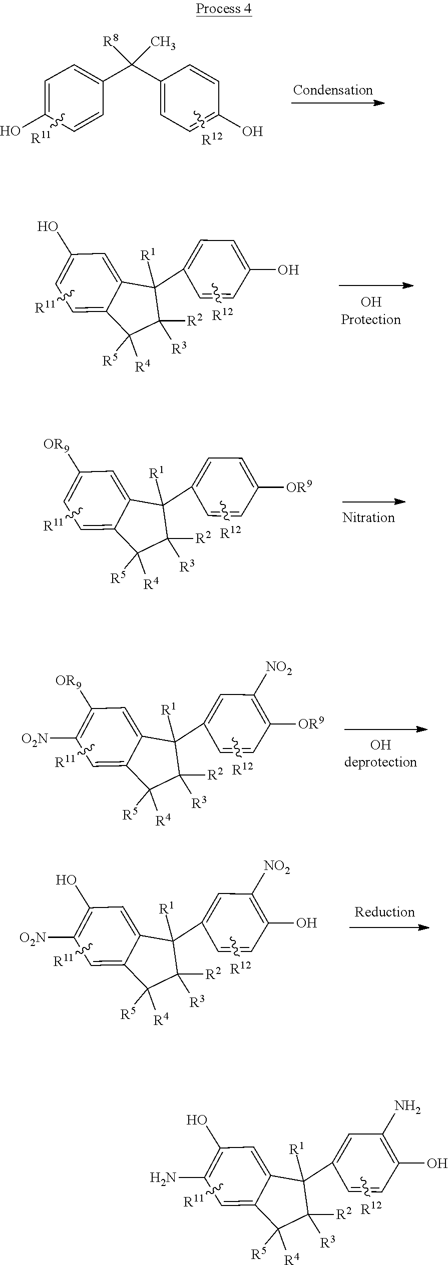

Log P is an important molecular physical property that influences a wide range of parameters in formulations. Log P is the measure of the preference of a compound to be dissolved in either water or an organic solvent (such as octanol) when uncharged. More technically, it is the logarithm of the partition coefficient (P) of a molecule between an aqueous and lipophilic phase. Log P predicts the partition coefficient, a measure of hydrophobicity, from structure. Although log P is a constant, its value is dependent on the choice of the organic partitioning solvent and, to a lesser degree, on the conditions of measurement. ACD/Labs' log P algorithms specifically calculate partitioning between octan-1-ol and water—the most commonly used system. An example of Log P of bis-o-aminophenol are: 2,2-Bis(3-amino-4-hydroxyphenyl)hexafluoropropane (log P 0.74), 2,2-Bis(3-amino-4-hydroxylphenyl)propane (log P 1.35), 9,9-Bis(3-amino-4 hydroxyphenyl)fluorene (Log P 2.71), Bis(3-amino-4-hydroxyphenyl) Sulfone (1.03) and 6-amino-3-(3′-amino-4′-hydroxyphenyl)-1,1,3-trimethyl-2,3-dihydro-1H-inden-5-ol (Indane bis-o-aminophenol) (Log P 2.61).

(5) or (6)-amino-1-(4-aminophenyl)-1,3,3-trimethylindane (DAPI) are diamine monomers which form solvent soluble polyimides with even rigid dianhydrides due to inherent asymmetry and non-planarity. At the same time, the DAPI based polyimide polymers are quite rigid which is evident from the high glass transition temperatures observed for these systems and thermoxidative stability due to absence of oxidatively susceptible benzylic hydrogens. The combination of log P, inherent asymmetry and non-planarity of indane bis-o-aminophenol compounds will provide good physicochemical properties of polyimide and polybenzoxazole polymers.

In other embodiments of this disclosure there are provided polymers produced by using at least one indane bis-o-aminophenol compound of the invention as a comonomer.

In some embodiments, the polymer may be comprised of at least one indane bis-o-aminophenol comonomer and a comonomer derived from a dianhydride, a comonomer derived from a diacid chloride, or a combination thereof.

The indane bis-o-aminophenol compounds described in this disclosure can provide fully imidized polyimides. Such polyimides can be used in photosensitive compositions which can be used in dielectric compositions suitable for producing dielectric layers that impart good reliability in microelectronic devices.

In some embodiments of this disclosure there are provided poly-o-hydroxyamides of Structure XI. These polymers are produced by utilizing at least one indane bis-o-aminophenol compound as a first comonomer and another diamine or mixture of diamines as a second comonomer, and a diacid chloride or mixture of diacid chlorides as a third comonomer:

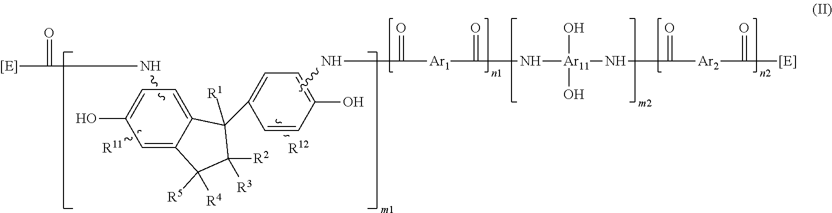

wherein each of R¹, R², R³, R⁴, and R⁵ independently, is a hydrogen atom, a substituted or unsubstituted C₁-C₁₂ alkyl, a partially halogen substituted or fully halogen substituted C₁-C₁₂ alkyl, a substituted or unsubstituted C₄-C₁₈ cycloalkyl, a substituted or unsubstituted C₆-C₂₂ aryl, or a substituted or unsubstituted C₅-C₂₂ heteroaryl; each of R¹¹ and R¹² independently is a hydrogen atom, a linear or branched C₁-C₄ alkyl, a partially halogen substituted or fully halogen substituted C₁-C₄ alkyl, a C₅-C₁₂ cycloalkyl, a C₆-C₁₈ aryl, a C₅-C₁₈ heteroaryl group, a C₁-C₄ alkoxy group or a halogen atom; Ar₁ and Ar₂ are each independently a divalent aromatic, aliphatic, or heterocyclic group, or mixtures thereof; Ar₁₁ is a divalent aromatic, aliphatic, or heterocyclic group or siloxane group; E is an end-capping group; n₁ is an integer from 5 to 200; m₁ is an integer from 5 to 200; m₂ is an integer from 0 to 200 and n₂ is an integer from 0 to 200.

As used herein, “end-capping group” refers to the reaction products of amino end groups of poly-o-hydroxyamides with monoanhydride compounds. Monoanhydride compounds, when added to the polymerization system, facilitate termination of a poly-o-hydroxyamide chain, thereby limiting polymer chain growth.

In some embodiments, Ar₁ and Ar₂ include the following moieties:

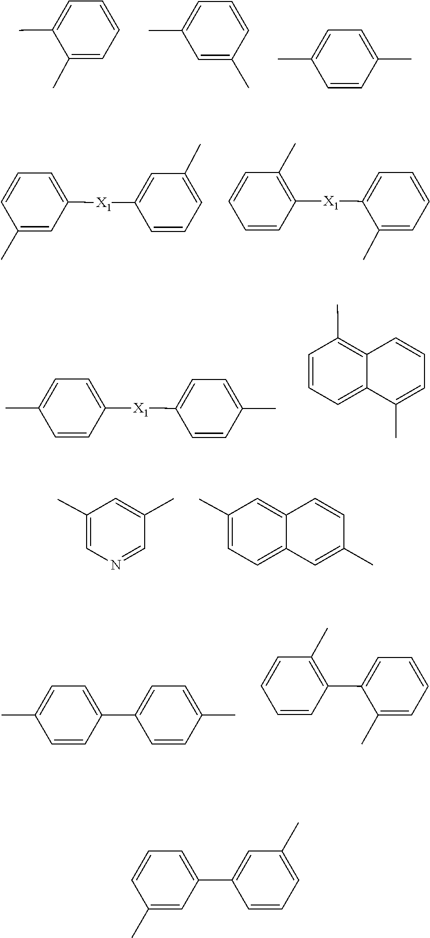

in which X₁ is —C(O)—C(O)—, —C(O)O—, or —(CH₂)ₚ—Si(Z)₂—O—Si(Z)₂—(CH₂)ₚ—, Z is H or C₁-C₆ alkyl and p is an integer from 1 to 6. In some embodiments, the poly-o-hydroxyamide can contain one or more different Ar₁ and Ar₂ groups.

In some embodiments, Ar₁₁ includes the following moieties:

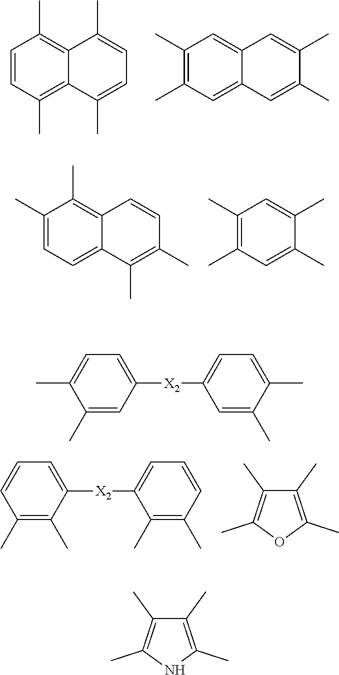

in which X₂ is —O—, —S—, —C(CF₃)₂—, —C(CH₃)₂—, —CH₂—, —SO₂—, —NHCO—, —C(O)—, —C(O)—C(O)—, —C(O)O—, or —(CH₂)ₘ—Si(Z)₂—O—Si(Z)₂—(CH₂)ₘ—, Z is H or C₁-C₆ alkyl and m is an integer from 1 to 6.

In certain embodiments, the disclosure provides poly-o-hydroxyamide polymers that can be used in positive tone or negative tone photosensitive compositions, wherein the positive photosensitive resin composition and the negative photosensitive resin composition are soluble in an aqueous alkaline solution and are capable of forming a fine pattern with high resolution. The compositions have good mechanical properties even when cured at low temperature.

In some embodiments, the disclosure provides compositions comprising the dielectric film-forming compositions described herein including (a) at least one polymer comprising at least one indane bis-o-aminophenol compound as diamine comonomer; a comonomer derived from a dianhydride, or a comonomer derived from a diacid dichloride, or a combination thereof, thereby providing a dielectric layer, wherein, when the dielectric layer is present in a microelectronic device, the modified dielectric layer imparts good reliability to the device.

The present disclosure is illustrated in more detail with reference to the following examples, which are for illustrative purposes and should not be construed as limiting the scope of the present disclosure.

### Examples

### Synthesis of 4-Isopropenyl phenol (IPP) Oligomers

Bisphenol-A (50 g) is dissolved and stirred in concentrated sulfuric acid (300 g) at 25° C. After, bisphenol-A is completely dissolved (about 30 minutes), the solution is poured slowly into 3 L of ice-water while vigorously stirring. A sticky solid with slight orange color is formed instantaneously. After complete addition, the temperature of the solution is allowed to rise to room temperature and the solution is stirred for 60 minutes. The sticky solid precipitates are filtered, and after crystallization from toluene, the product, 35 g of IPP-oligomers (70% yield) is obtained.

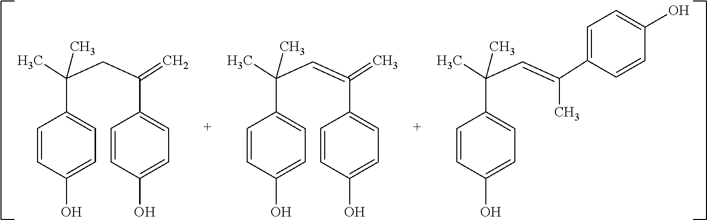

### Preparation of 3-(4′Hydroxyphenyl)-1,1,3-trimethyl-5-Indanol

IPP-oligomers, (25 g, prepared from BPA) are dissolved in 100 ml formic acid and the solution is stirred for a period of one hour until solid precipitates are formed. After filtration and drying in the oven, 17.5 g (75% yield based on BPA, 99% recovery by IPP-dimers) product is collected.

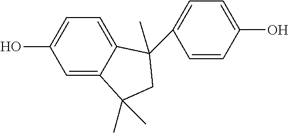 [SMILES: CC1(C)CC(C)(c2ccc(O)cc2)c2ccc(O)cc21]

### Preparation of 6-nitro-3-(3′-nitro-4′hydroxyphenyl)-1,1,3-trimethyl-2,3-dihydro-1H-Inden-5-ol

To a 3-neck round bottom flask equipped with a mechanical stirrer and a reflux condenser is added 26.8 grams of 3-(4′-Hydroxyphenyl)-1,1,3-trimethyl-5-indanolin 200 ml of acetic acid. The mixture is warmed to 50 degrees centigrade and then 175 mL (2.3 molar equivalent) of nitric acid (specific gravity: 1.38) is added dropwise to the solution for about 1 hour at 50 degrees centigrade. Thereafter, the solution is left at 50 degrees centigrade for 1 hour. Then, 150 mL of water is added to the solution while adjusting a slurry concentration for sufficiently depositing the crystallized substance. The solid is filtered and washed with 50 percent aqueous methanol and then dried in air. Finally, the crude mixture is crystallized from 95 percent ethanol. The product, 34 g of dinitro compound, 6-nitro-3-(3′-nitro-4′-hydroxyphenyl)-1,1,3-trimethyl-2,3-dihydro-1H-inden-5-ol (94% yield) is obtained.

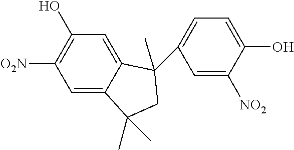 [SMILES: CC1(C)CC(C)(c2ccc(O)c([N+](=O)[O-])c2)c2cc(O)c([N+](=O)[O-])cc21]

### Preparation of 6-amino-3-(3′-amino-4′-hydroxyphenyl)-1,1,3-trimethyl-2,3-dihydro-1H-Inden-5-ol (Indane bis-o-aminophenol)

To a pressure reactor equipped with a mechanical stirrer is added 30 grams of 6-nitro-3-(3′-nitro-4′-hydroxyphenyl)-1,1,3-trimethyl-2,3-dihydro-1H-inden-5-ol, 0.44 grams of 5 percent Pt/C, 0.044 grams of H₃PO₂, 0.07 grams of [VO(acac)₂], and 200 grams of toluene. The reaction is performed under 5 bar H₂ pressure at 100 degrees centigrade. The solution is concentrated to dryness after completion of reaction. The crude solid is purified by recrystallization from aqueous ethanol. The product, 20 g of 6-amino-3-(3′-amino-4′-hydroxyphenyl)-1,1,3-trimethyl-2,3-dihydro-1H-inden-5-ol (80% yield) is obtained.

### Melting Point of Indane Bis-o-Aminophenol

The melting point of Indane bis-o-aminophenol was 228° C. measured by DSC (differential scanning calorimetry).

### NMR Analysis of Indane Bis-o-Aminophenol

¹H NMR (400 MHz): DMSO d₆, δ 0.97 (s, 3H), 1.20 (s, 3H), 1.44 (s, 3H), 1.94 (d, 1H), 2.18 (d, 1H), 4.34 (s, 4H, NH₂), 6.19 (q, 1H), 6.30 (s, 1H), 6.39 (s, 1H), 6.45 (q, 1H), 8.66 (d, 2H, OH).

¹³C NMR: 101 MHz, DMSO d₆, δ 31.1 (s), 31.5 (s), 31.7 (s), 42.3 (s), 49.6 (s), 60.1 (s), 108.2 (s), 110.3 (s), 113.7 (d), 114.9 (s), 135.9 (d), 137.7 (s), 1420 (d), 143.1 (s), 143.7 (s).

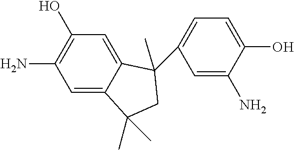 [SMILES: CC1(C)CC(C)(c2ccc(O)c(N)c2)c2cc(O)c(N)cc21]

While the invention has been described in detail with reference to certain embodiments thereof, it will be understood that modifications and variations are within the spirit and scope of that which is described and claimed.

What is claimed is:

1. An indane bis-o-aminophenol compound having formula Ia: 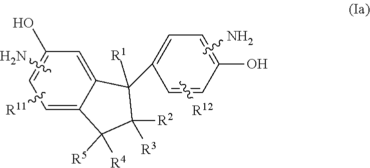
wherein each of R¹, R², R³, R⁴, and R⁵ independently, is a hydrogen atom, a substituted or unsubstituted C₁-C₁₂ alkyl, a partially halogen substituted or fully halogen substituted C₁-C₁₂ alkyl, a substituted or unsubstituted C₄-C₁₈ cycloalkyl, a substituted or unsubstituted C₆-C₂₂ aryl, or a substituted or unsubstituted C₅-C₂₂ heteroaryl; each of R¹¹ and R¹² is independently a hydrogen atom, a linear or branched C₁-C₄ alkyl, a partially halogen substituted or fully halogen substituted C₁-C₄alkyl, a C₄-C₁₂ cycloalkyl, a C₆-C₁₈ aryl, a C₅-C₁₈ heteroaryl group, a C₁-C₄ alkoxy group or a halogen atom.

2. The indane bis-o-aminophenol compound of claim 1, wherein each of R¹, R², R³, R⁴, and R⁵ each independently, is a hydrogen atom, a substituted or unsubstituted C₁-C₁₂ alkyl, or a substituted or unsubstituted C₄-C₁₈ cycloalkyl; each of R¹¹ and R¹² independently, is a hydrogen atom, a hydrogen atom, a linear or branched C₁-C₄ alkyl, a substituted or unsubstituted C₄-C₁₂ cycloalkyl, or a C₁-C₄ alkoxy group.

3. The indane bis-o-aminophenol compound of claim 1, wherein R¹ or R⁴ each independently is a substituted or unsubstituted C₁-C₁₂ alkyl; R², R³, and R⁵, are independently a hydrogen atom, a substituted or unsubstituted C₁-C₁₂ alkyl, or a substituted or unsubstituted C₄-C₁₈ cycloalkyl; and each of R¹¹ and R¹² is independently a hydrogen atom, a linear or branched C₁-C₄ alkyl, a substituted or unsubstituted C₄-C₁₂ cycloalkyl, or a substituted or unsubstituted C₁-C₄ alkoxy group.

4. The indane bis-o-aminophenol compound of claim 1, wherein R¹ or R⁴ each independently is a substituted or unsubstituted C₁-C₁₂ alkyl group; R² and R³ is independently a hydrogen atom; R⁵ is a hydrogen atom, a substituted or unsubstituted C₁-C₁₂ alkyl, or a substituted or unsubstituted C₄-C₁₈ cycloalkyl; each of R¹¹ and R¹² independently, is a hydrogen atom, a linear or branched C₁-C₄ alkyl, a C₄-C₁₂ cycloalkyl, or a C₁-C₄ alkoxy group.

5. The indane bis-o-aminophenol compound of claim 1, wherein the indane bis-o-aminophenol compound is selected from the following compounds:

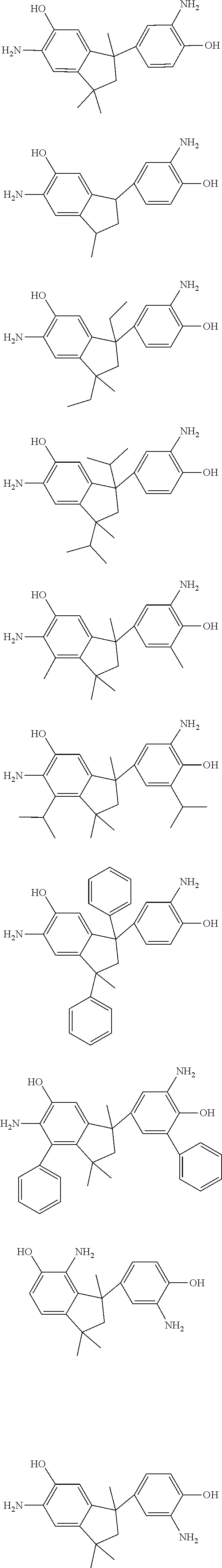

6. A polyimide polymer, comprising the reaction product of components (a), (b), and (c), wherein components (a), (b), and (c), are:
(a) at least one indane bis-o-aminophenol compound selected from the group consisting of an indane bis-o-aminophenol compound of claim 1,
(b) at least one other diamine; and
(c) at least one tetracarboxylic acid dianhydride.

7. A polymer comprising a poly-o-hydroxyamide represented by the following general formula (II): 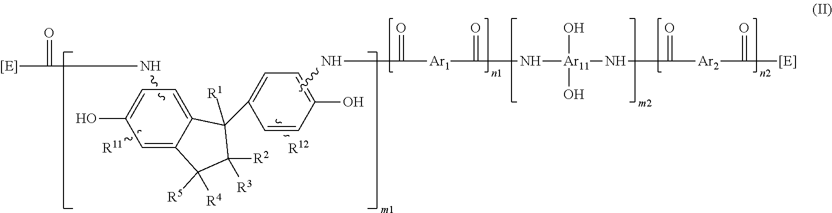
wherein each of R¹, R², R³, R⁴, and R⁵ independently, is a hydrogen atom, a substituted or unsubstituted C₁-C₁₂ alkyl, a partially halogen substituted or fully halogen substituted C₁-C₁₂ alkyl, a substituted or unsubstituted C₄-C₁₈ cycloalkyl, a substituted or unsubstituted C₆-C₂₂ aryl, or a substituted or unsubstituted C₅-C₂₂ heteroaryl; each of R¹¹ and R¹² independently is a hydrogen atom, a linear or branched C₁-C₄ alkyl, a partially halogen substituted or fully halogen substituted C₁-C₄ alkyl, a C₄-C₁₂ cycloalkyl, a C₆-C₁₈ aryl, C₅-C₁₈ heteroaryl group, a C₁-C₄ alkoxy group or a halogen atom; Ar₁ and Ar₂ are a divalent aromatic, aliphatic, or heterocyclic group, or mixtures thereof; Ar₁₁ is a divalent aromatic, aliphatic, or heterocyclic group or siloxane group; E is an end-capping group; m₁ is an integer from 5 to 200; n₁ is an integer from 5 to 200; m₂ is an integer from 0 to 200 and n₂ is an integer from 0 to 200.
